(19) United States
(12) Patent Application Publication
(10) Pub. No.: US 2024/0382642 A1
(43) Pub. Date: Nov. 21, 2024

(54) ELECTRONIC FRAGRANCE DIFFUSER SYSTEM AND METHOD

(71) Applicant: ATMOS FRAGRANCE, INC., SALT LAKE CITY, UT (US)

(72) Inventors: Kristen Knight, Lehi, UT (US); Tyson Andrus, Millcreek, UT (US); Wayne Moorehead, Salt Lake City, UT (US)

(21) Appl. No.: 18/669,483

(22) Filed: May 20, 2024

Related U.S. Application Data
(60) Provisional application No. 63/467,781, filed on May 19, 2023.

(51) Int. Cl. A61L 9/03; A61L 9/12; A61L 9/14

(52) U.S. Cl. CPC A61L 9/035; A61L 9/125; A61L 9/14; A61L 2209/11; A61L 2209/132; A61L 2209/135; A61L 2209/21

(57) ABSTRACT

A fragrance diffuser includes a housing configured to house at least two fragrance containers each containing a corresponding one of at least two fragrances. The fragrance diffuser also includes at least two dispensers each configured to dispense a corresponding one of the at least two fragrances from a corresponding one of the at least two fragrance containers when the at least two fragrance containers are housed by the housing. The fragrance diffuser further includes an electronic controller configured to operate the fragrance diffuser in at least one of a timed rotation mode, a shuffle mode, a blend mode, or a run-through mode.

### CROSS-REFERENCE TO RELATED APPLICATION

This application claims the benefit of U.S. Provisional Patent Application No. 63/467,781, filed May 19, 2023, which is incorporated herein by reference in its entirety.

### FIELD

This disclosure relates generally to fragrance diffusers, and more particularly to electronically-controlled fragrance diffusers.

### BACKGROUND

Electronically-controlled fragrance diffusers enable diffusion of fragrances into a space via electronic means. Some electronic fragrance diffusers enable a user to customize when a fragrance is diffused. However, the customization of the diffusion schedule for existing electronic fragrance diffusers is limited. Moreover, existing electronic diffusers do not properly account for olfactory fatigue (e.g., noseblindness) commonly associated with prolonged exposure to odors. Additionally, current electronic diffusers are limited to diffusing pre-set fragrances and do not allow for customization of fragrances by a user.

### SUMMARY

The subject matter of the present application has been developed in response to the present state of the art, and in particular, in response to the problems and needs of conventional systems and methods for diffusing fragrances. In view of the foregoing, the subject matter of the present application has been developed to provide an electronic fragrance diffuser system and method that overcome many of the shortcomings of the prior art.

The following is a non-exhaustive list of examples, which may or may not be claimed, of the subject matter, disclosed herein.

Disclosed herein is a fragrance diffuser that includes a housing configured to house at least two fragrance containers each containing a corresponding one of at least two fragrances. The fragrance diffuser also includes at least two dispensers each configured to dispense a corresponding one of the at least two fragrances from a corresponding one of the at least two fragrance containers when the at least two fragrance containers are housed by the housing. The fragrance diffuser further includes an electronic controller configured to operate the fragrance diffuser in at least one of a timed rotation mode, a shuffle mode, a blend mode, or a run-through mode. When the at least two fragrance containers are housed by the housing, in the timed rotation mode, the electronic controller controls the at least two dispensers so that a first one of the at least two dispensers dispenses a first one of the at least two fragrances for a first rotation period, and, so that after the first rotation period, a second one of the at least two dispensers dispenses a second one of the at least two fragrances for a second rotation period. When the at least two fragrance containers are housed by the housing, in the shuffle mode, the electronic controller controls the at least two dispensers so that the first one of the at least two dispensers dispenses the first one of the at least two fragrances for a first shuffle period. The first one of the at least two dispensers is randomly selected from the at least two dispensers via a random order generator of the electronic controller. After the first shuffle period, the second one of the at least two dispensers dispenses the second one of the at least two fragrances for a second shuffle period equal to the first shuffle period. When the at least two fragrance containers are housed by the housing, in the blend mode, the electronic controller controls the at least two dispensers so that, concurrently, the first one of the at least two dispensers dispenses the first one of the at least two fragrances and the second one of the at least two dispensers dispenses the second one of the at least two fragrances, and so that the first one of the at least two fragrances, after being dispensed, blends with the second one of the at least two fragrances, after being dispensed, to form a blend of fragrances. When the at least two fragrance containers are housed by the housing, in the run-through mode, the electronic controller controls the at least two dispensers so that the first one of the at least two dispensers dispenses the first one of the at least two fragrances until the first one of the at least two fragrances is depleted from the first one of the at least two dispensers, and so that after the first one of the at least two fragrances is depleted from the first one of the at least two dispensers, the second one of the at least two dispensers automatically dispenses the second one of the at least two fragrances until the second one of the at least two fragrances is depleted from the second one of the at least two dispensers. The preceding subject matter of this paragraph characterizes example 1 of the present disclosure.

The electronic controller is configured to operate the fragrance diffuser in at least two of the timed rotation mode, the shuffle mode, the blend mode, or the run-through mode. The preceding subject matter of this paragraph characterizes example 2 of the present disclosure, wherein example 2 also includes the subject matter according to example 1, above.

The electronic controller is configured to operate the fragrance diffuser in at least three of the timed rotation mode, the shuffle mode, the blend mode, or the run-through mode. The preceding subject matter of this paragraph characterizes example 3 of the present disclosure, wherein example 3 also includes the subject matter according to example 2, above.

The electronic controller is configured to operate the fragrance diffuser in all four of the timed rotation mode, the shuffle mode, the blend mode, and the run-through mode. The preceding subject matter of this paragraph characterizes example 4 of the present disclosure, wherein example 4 also includes the subject matter according to example 3, above.

The electronic controller is configured to operate the fragrance diffuser in at least the timed rotation mode. The preceding subject matter of this paragraph characterizes example 5 of the present disclosure, wherein example 5 also includes the subject matter according to any of examples 1-4, above.

Each one of the first rotation period and the second rotation period is equal to or less than a time period associated with olfactory fatigue. The preceding subject matter of this paragraph characterizes example 6 of the present disclosure, wherein example 6 also includes the subject matter according to example 5, above.

The first rotation period and the second rotation period is a user-selected one of a plurality of rotation periods. The preceding subject matter of this paragraph characterizes example 7 of the present disclosure, wherein example 7 also includes the subject matter according to any of examples 5-6, above.

The first one of the at least two dispensers is a user-selected one of the at least two dispensers. The preceding subject matter of this paragraph characterizes example 8 of the present disclosure, wherein example 8 also includes the subject matter according to any of examples 5-7, above.

In the time rotation mode, the electronic controller is configured to continuously switch between dispensing the at least two fragrances for a scheduled period of time. The preceding subject matter of this paragraph characterizes example 9 of the present disclosure, wherein example 9 also includes the subject matter according to any of examples 5-8, above.

The second rotation period is equal to the first rotation period. The preceding subject matter of this paragraph characterizes example 10 of the present disclosure, wherein example 10 also includes the subject matter according to any of examples 5-9, above.

The second rotation period is different than the first rotation period. The preceding subject matter of this paragraph characterizes example 11 of the present disclosure, wherein example 11 also includes the subject matter according to any of examples 5-9, above.

The electronic controller is configured to operate the fragrance diffuser in at least the shuffle mode. The preceding subject matter of this paragraph characterizes example 12 of the present disclosure, wherein example 12 also includes the subject matter according to any of examples 1-11, above.

Each one of the first shuffle period and the second shuffle period is equal to or less than a time period associated with olfactory fatigue. The preceding subject matter of this paragraph characterizes example 13 of the present disclosure, wherein example 13 also includes the subject matter according to example 12, above.

The first shuffle period and the second shuffle period is a user-selected one of a plurality of shuffle periods. The preceding subject matter of this paragraph characterizes example 14 of the present disclosure, wherein example 14 also includes the subject matter according to any of examples 12-13, above.

In the shuffle mode, the electronic controller is configured to continuously switch between dispensing the at least two fragrances in a random order for a scheduled period of time. The preceding subject matter of this paragraph characterizes example 15 of the present disclosure, wherein example 15 also includes the subject matter according to any of examples 12-14, above.

In the shuffle mode, the fragrance diffuser is configured to dispense the at least two fragrances in multiple cycles, and wherein an order of the at least two fragrances in a first one of the multiple cycles is different than the order of the at least two fragrances in a second one of the multiple cycles. The preceding subject matter of this paragraph characterizes example 16 of the present disclosure, wherein example 16 also includes the subject matter according to example 15, above.

The electronic controller is configured to operate the fragrance diffuser in at least the blend mode. The preceding subject matter of this paragraph characterizes example 17 of the present disclosure, wherein example 17 also includes the subject matter according to any of examples 1-16, above.

The first one of the at least two fragrances makes up a first percentage of the blend of fragrances. The second one of the at least two fragrances makes up a second percentage of the blend of fragrances. Each one of the first percentage and the second percentage is a user-selected one of a plurality of percentages. The preceding subject matter of this paragraph characterizes example 18 of the present disclosure, wherein example 18 also includes the subject matter according to example 17, above.

When operating in the blend mode, the electronic controller is configured to diffuse the first one of the at least two fragrances at a first intensity and to diffuse the second one of the at least two fragrances at a second intensity. When operating in the blend mode, the first intensity of the first one of the at least two fragrances and the second intensity of the second one of the at least two fragrances are combined to equal a user-selected one of a plurality of intensities. The preceding subject matter of this paragraph characterizes example 19 of the present disclosure, wherein example 19 also includes the subject matter according to example 18, above.

The electronic controller further includes a memory. The blend of fragrances, including the first percentage of the first one of the at least two fragrances and the second percentage of the second one of the at least two fragrances, is stored in the memory after the first percentage and the second percentage are selected by a user. The preceding subject matter of this paragraph characterizes example 20 of the present disclosure, wherein example 20 also includes the subject matter according to any of examples 18-19, above.

The blend of fragrances stored in the memory is re-selectable by a user. The preceding subject matter of this paragraph characterizes example 21 of the present disclosure, wherein example 21 also includes the subject matter according to example 20, above.

The fragrance diffuser is configured to share with others, across a computer network, the blend of fragrances, including the first percentage of the first one of the at least two fragrances and the second percentage of the second one of the at least two fragrances, selected by the user. The preceding subject matter of this paragraph characterizes example 22 of the present disclosure, wherein example 22 also includes the subject matter according to any of examples 20-21, above.

In the blend mode, the electronic controller is further configured to control the at least two dispensers so that concurrently, the first one of the at least two dispensers dispenses the first one of the at least two fragrances and the second one of the at least two dispensers dispenses the second one of the at least two fragrances form a second blend of fragrances different than the blend of fragrances. In the blend mode, the electronic controller is configured to continuously switch between dispensing the blend of fragrances, for a blend period, and the second blend of fragrances, for the blend period, for a scheduled period of time. The preceding subject matter of this paragraph characterizes example 23 of the present disclosure, wherein example 23 also includes the subject matter according to any of examples 17-22, above.

The blend period is equal to or less than a time period associated with olfactory fatigue. The preceding subject matter of this paragraph characterizes example 24 of the present disclosure, wherein example 24 also includes the subject matter according to example 23, above.

The electronic controller is configured to operate the fragrance diffuser in at least the run-through mode. The preceding subject matter of this paragraph characterizes example 25 of the present disclosure, wherein example 25 also includes the subject matter according to any of examples 1-24, above.

The housing is configured to house at least three fragrance containers each containing a corresponding one of at least three fragrances. The fragrance diffuser includes at least three dispensers each configured to dispense a corresponding one of the at least three fragrances from a corresponding one of the at least three fragrance containers when the at least three fragrance containers are housed by the housing. When the at least three fragrance containers are housed by the housing, in the timed rotation mode, the electronic controller controls the at least three dispensers so that the first one of the at least three dispensers dispenses the first one of the at least three fragrances for the first rotation period. After the first rotation period, the second one of the at least three dispensers dispenses the second one of the at least three fragrances for the second rotation period. After the second rotation period, a third one of the at least three dispensers dispenses a third one of the at least three fragrances for a third rotation period equal to the second rotation period. When the at least three fragrance containers are housed by the housing, in the shuffle mode, the electronic controller controls the at least three dispensers so that the first one of the at least three dispensers dispenses the first one of the at least three fragrances for the first shuffle period. The first one of the at least three dispensers is randomly selected from the at least three dispensers via the random order generator of the electronic controller. After the first shuffle period, the second one of the at least three dispensers dispenses the second one of the at least three fragrances for the second shuffle period. After the second shuffle period, a third one of the at least three dispensers dispenses a second one of the at least three fragrances for a third shuffle period equal to the second shuffle period. When the at least three fragrance containers are housed by the housing, in the blend mode, the electronic controller controls the at least three dispensers so that concurrently, the first one of the at least three dispensers dispenses the first one of the at least three fragrances, the second one of the at least three dispensers dispenses the second one of the at least three fragrances, and a third one of the at least three dispensers dispenses a third one of the at least three fragrances. The first one of the at least three fragrances, after being dispensed, blends with the second one of the at least two fragrances, after being dispensed, and blends with the third one of the at least three fragrances, to form the blend of fragrances. When the at least three fragrance containers are housed by the housing, in the run-through mode, the electronic controller controls the at least three dispensers so that the first one of the at least three dispensers dispenses the first one of the at least three fragrances until the first one of the at least three fragrances is depleted from the first one of the at least three dispensers. After the first one of the at least three fragrances is depleted from the first one of the at least three dispensers, the second one of the at least three dispensers automatically dispenses the second one of the at least three fragrances until the second one of the at least three fragrances is depleted from the second one of the at least three dispensers. After the second one of the at least three fragrances is depleted from the second one of the at least three dispensers, a third one of the at least three dispensers automatically dispenses a third one of the at least three fragrances until the third one of the at least three fragrances is depleted from the third one of the at least three dispensers. The preceding subject matter of this paragraph characterizes example 26 of the present disclosure, wherein example 26 also includes the subject matter according to any of examples 1-25, above.

The electronic controller includes a schedule module configured to operate the fragrance diffuser in each one of the at least one of the timed rotation mode, the shuffle mode, the blend mode, or the run-through mode according to a user-selected one of a plurality of schedules. The preceding subject matter of this paragraph characterizes example 27 of the present disclosure, wherein example 27 also includes the subject matter according to any of examples 1-26, above.

The electronic controller is configured to diffuse the first one of the at least two fragrances and the second one of the at least two fragrances according to a user-selected one of a plurality of intensities when the fragrance diffuser is operating in the at least one of the timed rotation mode, the shuffle mode, the blend mode, or the run-through mode. The preceding subject matter of this paragraph characterizes example 28 of the present disclosure, wherein example 28 also includes the subject matter according to any of examples 1-27, above.

Additionally disclosed herein is a method of diffusing fragrances. The method includes at least one of a timed rotation step, a shuffle step, a blend step, or a run-through step. The timed rotation step includes electronically controlling at least two dispensers so that a first one of the at least two dispensers dispenses a first one of at least two fragrances for a first rotation period, and so that after the first rotation period, a second one of the at least two dispensers dispenses a second one of the at least two fragrances for a second rotation period. The shuffle step includes electronically controlling the at least two dispensers so that the first one of the at least two dispensers dispenses the first one of the at least two fragrances for a first shuffle period. The first one of the at least two dispensers is randomly selected from the at least two dispensers via a random order generator of the electronic controller. After the first shuffle period, the second one of the at least two dispensers dispenses the second one of the at least two fragrances for a second shuffle period equal to the first shuffle period. The blend step includes electronically controlling the at least two dispensers so that concurrently, the first one of the at least two dispensers dispenses the first one of the at least two fragrances and the second one of the at least two dispensers dispenses the second one of the at least two fragrances, and the first one of the at least two fragrances, after being dispensed, blends with the second one of the at least two fragrances, after being dispensed, to form a blend of fragrances. The run-through step includes electronically controlling the at least two dispensers so that the first one of the at least two dispensers dispenses the first one of the at least two fragrances until the first one of the at least two fragrances is depleted from the first one of the at least two dispensers. After the first one of the at least two fragrances is depleted from the first one of the at least two dispensers, the second one of the at least two dispensers automatically dispenses the second one of the at least two fragrances until the second one of the at least two fragrances is depleted from the second one of the at least two dispensers. The preceding subject matter of this paragraph characterizes example 29 of the present disclosure.

The method includes at least two of the timed rotation step, the shuffle step, the blend step, or the run-through step. The preceding subject matter of this paragraph characterizes example 30 of the present disclosure, wherein example 30 also includes the subject matter according to example 29, above.

The method includes at three of the timed rotation step, the shuffle step, the blend step, or the run-through step. The preceding subject matter of this paragraph characterizes example 31 of the present disclosure, wherein example 31 also includes the subject matter according to any of examples 29-30, above.

The method includes all four of the timed rotation step, the shuffle step, the blend step, and the run-through step. The preceding subject matter of this paragraph characterizes example 32 of the present disclosure, wherein example 32 also includes the subject matter according to any of examples 29-31, above.

The method includes the timed rotation step. Each one of the first rotation period and the second rotation period is equal to or less than a time period associated with olfactory fatigue. The time rotation step further includes selecting the first rotation period and the second rotation period from a plurality of rotation periods. The preceding subject matter of this paragraph characterizes example 33 of the present disclosure, wherein example 33 also includes the subject matter according to any of examples 29-32, above.

The method includes the shuffle step. each one of the first shuffle period and the second shuffle period is equal to or less than a time period associated with olfactory fatigue. The shuffle step further includes selecting the first shuffle period and the second shuffle period from a plurality of shuffle periods. The shuffle step further includes, for a scheduled period of time, switching between at least a first cycle, where the at least two fragrances are dispensed in a first order, and at least a second cycle, where the at least two fragrances are dispensed in a second order different than the first order. The preceding subject matter of this paragraph characterizes example 34 of the present disclosure, wherein example 34 also includes the subject matter according to any of examples 29-33, above.

The method includes the blend step. The blend step further includes selecting a first percentage and a second percentage from a plurality of percentages. The first one of the at least two fragrances makes up the first percentage of the blend of fragrances. The second one of the at least two fragrances makes up the second percentage of the blend of fragrances. The preceding subject matter of this paragraph characterizes example 35 of the present disclosure, wherein example 35 also includes the subject matter according to any of examples 29-34, above.

The method further includes electronically storing the blend of fragrances to create a stored blend of fragrances. The blend step further includes selecting the stored blend of fragrances. The blend step additionally includes electronically controlling the at least two dispensers so that concurrently, the first one of the at least two dispensers dispenses the first one of the at least two fragrances and the second one of the at least two dispensers dispenses the second one of the at least two fragrances. The first one of the at least two fragrances, after being dispensed, blends with the second one of the at least two fragrances, after being dispensed, to form the stored blend of fragrances. The preceding subject matter of this paragraph characterizes example 36 of the present disclosure, wherein example 36 also includes the subject matter according to example 35, above.

The method further includes sharing the stored blend of fragrances with others across a computer network. The preceding subject matter of this paragraph characterizes example 37 of the present disclosure, wherein example 37 also includes the subject matter according to example 36, above.

The blend step further includes continuously switching between dispensing the blend of fragrances, for a blend period, and a second blend of fragrances, for the blend period, for a scheduled period of time. The second blend of fragrances includes a third percentage of the first one of the at least two fragrances and a fourth percentage of the second one of the at least two fragrances. At least one of the third percentage is different than the first percentage, or the fourth percentage is different than the second percentage. The preceding subject matter of this paragraph characterizes example 38 of the present disclosure, wherein example 38 also includes the subject matter according to any of examples 35-37, above.

The method includes the run-through step. The preceding subject matter of this paragraph characterizes example 39 of the present disclosure, wherein example 39 also includes the subject matter according to any of examples 29-38, above.

The method further includes selecting an intensity from a plurality of intensities. Each one of the timed rotation step, the shuffle step, the blend step, and the run-through step includes dispensing the first one of the at least two fragrances and dispensing the second one of the at least two fragrances according to the selected intensity. The preceding subject matter of this paragraph characterizes example 40 of the present disclosure, wherein example 40 also includes the subject matter according to any of examples 29-39, above.

The described features, structures, advantages, and/or characteristics of the subject matter of the present disclosure may be combined in any suitable manner in one or more examples and/or implementations. In the following description, numerous specific details are provided to impart a thorough understanding of examples of the subject matter of the present disclosure. One skilled in the relevant art will recognize that the subject matter of the present disclosure may be practiced without one or more of the specific features, details, components, materials, and/or methods of a particular example or implementation. In other instances, additional features and advantages may be recognized in certain examples and/or implementations that may not be present in all examples or implementations. Further, in some instances, well-known structures, materials, or operations are not shown or described in detail to avoid obscuring aspects of the subject matter of the present disclosure. The features and advantages of the subject matter of the present disclosure will become more fully apparent from the following description and appended numbered paragraphs, or may be learned by the practice of the subject matter as set forth hereinafter.

### BRIEF DESCRIPTION OF THE DRAWINGS

In order that the advantages of the subject matter may be more readily understood, a more particular description of the subject matter briefly described above will be rendered by reference to specific examples that are illustrated in the appended drawings. Understanding that these drawings, which are not necessarily drawn to scale, depict only certain examples of the subject matter and are not therefore to be considered to be limiting of its scope, the subject matter will be described and explained with additional specificity and detail through the use of the drawings, in which:

FIG. 1 is a perspective view of a fragrance diffuser, shown from a front of the fragrance diffuser, according to one or more examples of the present disclosure;

FIG. 2 is a perspective view of the fragrance diffuser of FIG. 1, shown from a rear of the fragrance diffuser, according to one or more examples of the present disclosure;

FIG. 3 is a perspective view of a fragrance diffuser of FIG. 1, shown from a front of the fragrance diffuser and shown with a cover removed, according to one or more examples of the present disclosure;

FIG. 4 is a schematic block diagram of a fragrance diffusing system, according to one or more examples of the present disclosure;

FIG. 5 is a schematic block diagram of an electronic controller of a fragrance diffuser having a timed rotation module, according to one or more examples of the present disclosure;

FIG. 6 is a schematic flow chart of a method of diffusing fragrances, in a timed rotation mode of operation, according to one or more examples of the present disclosure;

FIG. 7 is a schematic block diagram of an electronic controller of a fragrance diffuser having a shuffle module, according to one or more examples of the present disclosure;

FIG. 8 is a schematic flow chart of a method of diffusing fragrances, in a shuffle mode of operation, according to one or more examples of the present disclosure;

FIG. 9 is a schematic block diagram of an electronic controller of a fragrance diffuser having a blend module, according to one or more examples of the present disclosure;

FIG. 10 is a schematic flow chart of a method of diffusing fragrances, in a blend mode of operation, according to one or more examples of the present disclosure;

FIG. 11 is a schematic block diagram of an electronic controller of a fragrance diffuser having a run-through module, according to one or more examples of the present disclosure; and

FIG. 12 is a schematic flow chart of a method of diffusing fragrances, in a run-through mode of operation, according to one or more examples of the present disclosure.

### DETAILED DESCRIPTION

Reference throughout this specification to “one example,” “an example,” or similar language means that a particular feature, structure, or characteristic described in connection with the example is included in at least one example of the present disclosure. Appearances of the phrases “in one example,” “in an example,” and similar language throughout this specification may, but do not necessarily, all refer to the same example. Similarly, the use of the term “implementation” means an implementation having a particular feature, structure, or characteristic described in connection with one or more examples of the present disclosure, however, absent an express correlation to indicate otherwise, an implementation may be associated with one or more examples.

Described herein are examples of a system, fragrance diffuser, and a method for electronically-controlled diffusion of fragrances into a space (e.g., an interior space, such as a room). Examples of the present disclosure include a system having a fragrance diffuser that diffuses one or more fragrances according to a selected one of several different modes of operation. The modes of operation include a timed rotation mode, a shuffle mode, a blend mode, and a run-through mode. The selected mode of operation is selected by a user according to the user's preferences and/or according to a user-defined schedule. In some examples, the fragrance diffuser is configured to enable less than all of the different modes of operation. However, in other examples, the fragrance diffuser is configured to enable all of the different modes of operation. The user can schedule the fragrance diffuser to enable a mode of operation according to a user-defined schedule. For example, the fragrance diffuser can be scheduled to enable a certain one of the modes of operation during a selected time period and to enable a certain other one of the modes of operation during another selected time period so that the fragrance diffuser can switch from one mode of operation to another according to a user-defined schedule.

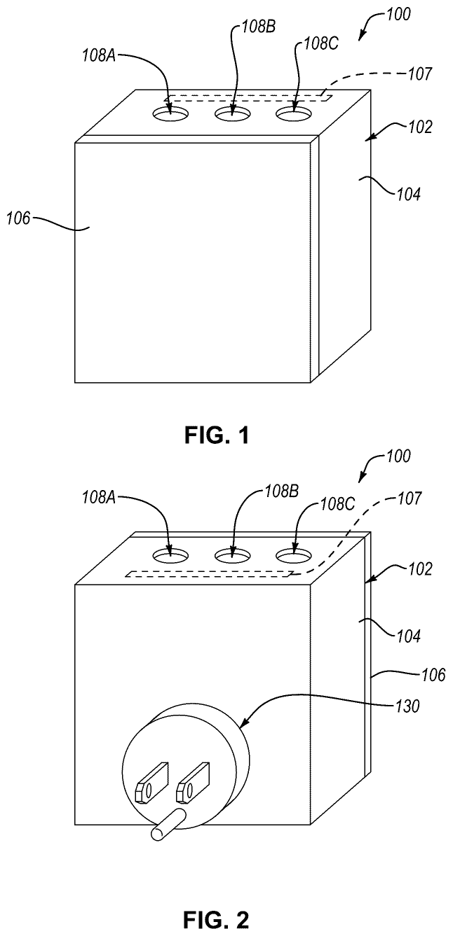

Referring to FIG. 1, according to one example, a fragrance diffuser 100 includes a housing 102. The housing 102 is configured to house and protect various internal components of the fragrance diffuser 100 as described below. In the illustrated example, the housing 102 includes a main body 104 and a cover 106. The main body 104 defines an interior cavity 110 and the cover 106 is attachable to the main body 104 to enclose the interior cavity 110, and detachable from the main body 104 to enable access to the interior cavity 110. In some examples, the cover 106 is attached to a front of the main body 104 and at least partially defines a front of the fragrance diffuser 100. The housing 102 can have any of various shapes, such as circular, rectangular, square (as shown), or the like. The housing 102 includes outlet apertures, such as first outlet aperture 108A, second outlet aperture 108B, and third outlet aperture 108C, which are open to the interior activity 110 and can be formed in the main body 104. In some examples, the outlet apertures are formed in a top of the housing 102.

The fragrance diffuser 100 optionally includes a visual communication device 107 coupled with the housing 102. The visual communication device 107 is configured to communicate to a user a status of the fragrance diffuser 100. For example, the visual communication device 107 can emit a light when a mode of operation of the fragrance diffuser 100 is enabled and one or more fragrances are being emitted by the fragrance diffuser 100. In certain examples, the visual communication device 107 emits light having different colors and/or at different blinking patterns, where each one of the colors and/or blinking patterns is associated with enablement of a corresponding one of multiple modes of operation, a fragrance level of two or more fragrances of the fragrance diffuser 100, and/or any of various other statuses of the fragrance diffuser 100. The visual communication device 107 includes one or more light emitters (e.g., an array of light-emitting diodes) and an at least partially transparent cover overlaying the light emitters. In some examples, the visual communication device 107 is fixed to (e.g., at least partially embedded in) the housing 102.

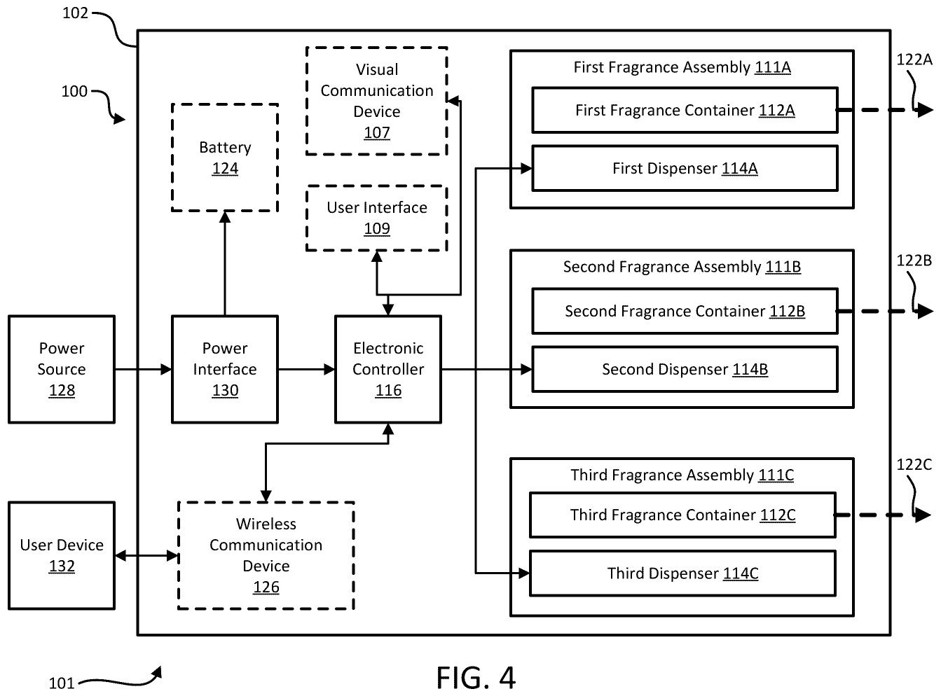

Referring to FIG. 2, in some examples, the fragrance diffuser 100 includes a power interface 130 that is configured to interface with a power source 128 (see, e.g., FIG. 4) to receive electrical power from the power source 128. The electrical power received from the power source 128 is used to power the electronic components of the fragrance diffuser 100. In the illustrated example, the power interface 130 is a standard electrical plug configured to be plugged into a standard electrical outlet (e.g., a wall outlet). Accordingly, the power interface 130 can include prongs that are insertable into openings in an electrical outlet to receive power from the electrical outlet. In other examples, the power interface 130 can be another type of power interface (such as a USB port, a USB-C port, an AC adaptor port, etc.). In one example, the power interface 130 is formed in a back portion of the fragrance diffuser 100, so that, when the power interface 130 is a standard electrical plug and is plugged into a wall outlet, the fragrance diffuser 100 is mounted to the wall.

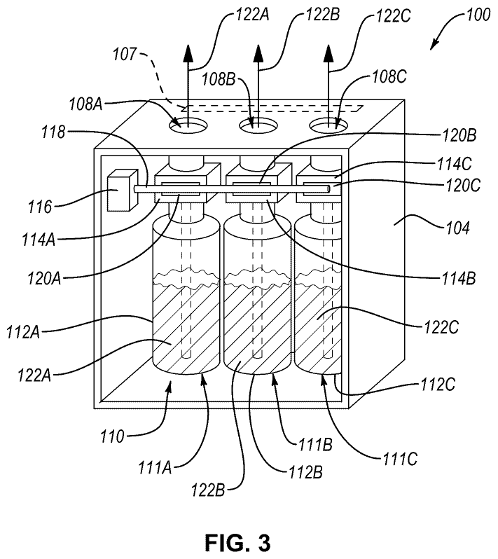

Referring to FIG. 3, the fragrance diffuser 100 further includes fragrance assemblies contained within the housing 102. For example, the fragrance diffuser 100 includes a first fragrance assembly 111A, a second fragrance assembly 111B, and a third fragrance assembly 111C. Each one of the fragrance assemblies includes a fragrance container and a dispenser. The fragrance container (e.g., cartridge) is configured to contain fragrance and the dispenser is configured to facilitate diffusion of the fragrance from the fragrance container into the interior space in which the fragrance diffuser 100 is located. The fragrance can be in any of various forms, such as a liquid, a solid, gel, etc., and the dispenser can be any of various types of dispensers corresponding to the form of the fragrance. According to other examples, the fragrance is soaked in a material, and the dispenser is a fan that enables diffusion of the liquid from the material via forced convection.

In the illustrated example, the fragrance is a liquid, and the dispenser is one of a heat-enabled dispenser, an ultrasonic water-based dispenser, an atomization dispenser, or a nebulization dispenser. More specifically, the first fragrance assembly 111A includes a first fragrance container 112A, in the form of a vial that contains a first fragrance 122A (e.g., in liquid form), and a first dispenser 114A, which dispenses (e.g., diffuses) the first fragrance 122A. The second fragrance assembly 111B includes a second fragrance container 112B, in the form of a vial that contains a second fragrance 122B (e.g., in liquid form), and a second dispenser 114B, which dispenses (e.g., diffuses) the second fragrance 122B. Likewise, the third fragrance assembly 111C includes a third fragrance container 112C, in the form of a vial that contains a third fragrance 122C (e.g., in liquid form), and a third dispenser 114C, which dispenses (e.g., diffuses) the third fragrance 122C. In some examples, the first fragrance 122A, the second fragrance 122B, and the third fragrance 122C are different fragrances. The first fragrance 122A is dispensed from the first dispenser 114A, through the first outlet aperture 108A, and into the interior space. The second fragrance 122B is dispensed from the second dispenser 114B, through the second outlet aperture 108B, and into the interior space. The third fragrance 122C is dispensed from the third dispenser 114C, through the third outlet aperture 108C, and into the interior space.

The fragrance diffuser 100 further includes an electronic controller 116 and a power bus 118. The electronic controller 116 is configured to control operation of the dispensers of the fragrance assemblies by communicating dispense commands to the dispensers via the power bus 118. In certain examples, each one of the dispensers includes an electrical interface that interfaces with the power bus 118 and facilitates the transmission of communications from the electronic controller 116 to the dispensers. In the illustrated example, the first dispenser 114A includes a first electrical interface 120A, the second dispenser 114B includes a second electrical interface 120B, and the third dispenser 114C includes a third electrical interface 120C.

Referring to FIG. 4, in some examples, the fragrance diffuser 100 can form part of a fragrance diffusing system 101, which additionally includes a user device 132 (e.g., one or more user devices). The fragrance diffuser 100 can further includes a battery 124 that is configured to store electrical power received from the power source 128. The battery 124 can function as a backup power source should the power source 128 be unable to deliver electrical power to the power interface 130. Or alternatively, the battery 124 can be a rechargeable battery, which is rechargeable via electrical power received from the power source 128 and functions as the primary battery source for the fragrance diffuser 100. In certain examples, the fragrance diffuser 100 does not include a power interface 130 and the battery 124 includes one or more replaceable batteries that supply the electrical power for operation of the fragrance diffuser 110 and are replaced when depleted.

In some examples, the fragrance diffuser 100 is wirelessly controllable by a user via wireless communication between the user device 132 of the fragrance diffusing system 101 and a wireless communication device 126 of the fragrance diffuser 100. The wireless communication device 126 includes any of various electronic components that facilitate the transmission and receipt of wireless signals. For example, the wireless communication device 126 can include a wireless transceiver configured to communicate with the user device 132 via a wireless communication protocol (e.g., cellular data network, wi-fi network, Bluetooth, infrared, etc.). The user device 132 can be any of various personal computer devices, such as laptop computers, cell phones, desktop computers, tablet computers, wearable devices, voice-activated platforms, etc. The user device 132 is configured to receive user inputs and communicate information, corresponding with the user inputs, to the electronic controller 116 via the wireless communication device 126. In addition, or alternative, to a user device 132, in certain examples, the fragrance diffuser 100 includes a user interface 109 that is configured to receive user inputs directly from the user. The user interface 109 can be one or more of a touchscreen, display, buttons, knobs, or other controls.

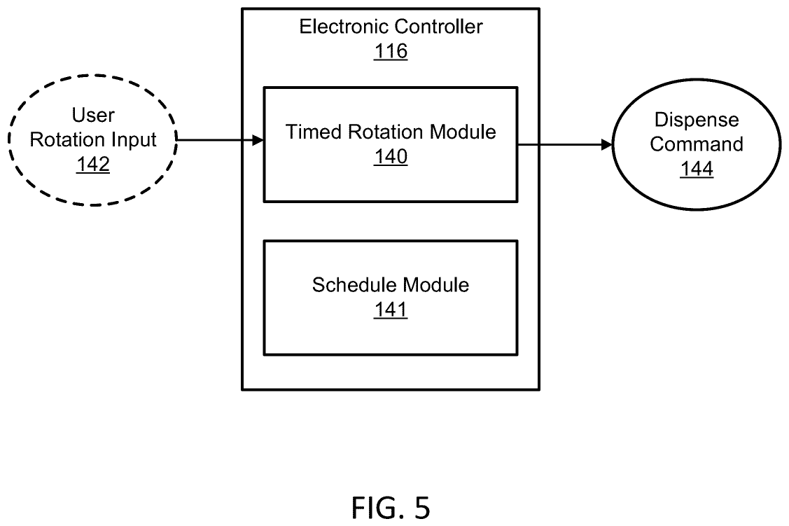

Referring to FIG. 5, according to some examples, the electronic controller 116 includes a timed rotation module 140 and a schedule module 141. The timed rotation module 140 is configured to enable a timed rotation mode of the fragrance diffuser 100 by generating a dispense command 144 (or dispense commands) pursuant to operations within the timed rotation mode. In one example, the dispense command 144 is embodied as an electrical power signal sent to an intended one of the dispensers. The dispense command 144 (e.g., electrical power signal) is received by an intended dispenser of a fragrance assembly and, in response to receipt of the dispense command 144, the dispenser dispenses the fragrance corresponding with the dispenser. When the dispense command 144 stops being sent to the intended dispenser, the intended dispenser stops dispensing the corresponding fragrance. The dispense command 144 is generated in response to user rotation input 142 from a user. The user rotation input 142 identifies a user's desire to enable (e.g., start) the timed rotation mode of the fragrance diffuser 100. The user rotation input 142 is received from the user via the user device 132 or the user interface 109. In some examples, the user rotation input 142 also includes a desired schedule for operating the fragrance diffuser 100 in the timed rotation mode. In response to receipt of a desired schedule (e.g., a user-selected schedule or a user-selected one of a plurality of schedules, which can include an infinite number of schedules), from the user rotation input 142, the schedule module 141 is operable to run the fragrance diffuser 100 in the timed rotation mode according to the desired schedule. For example, the desired schedule may include a start time and an end time and the schedule module 141 is configured to control the timed rotation module 140 to start and stop generating the dispense command 144 according to the desired schedule. Additionally, in certain examples, the user rotation input 142 also includes a desired intensity value (e.g., a user-selected intensity value or a user-selected one of a plurality of intensity values, which can include an infinite number of intensity values), which corresponds with a desired intensity of the fragrances diffused during the timed rotation mode.

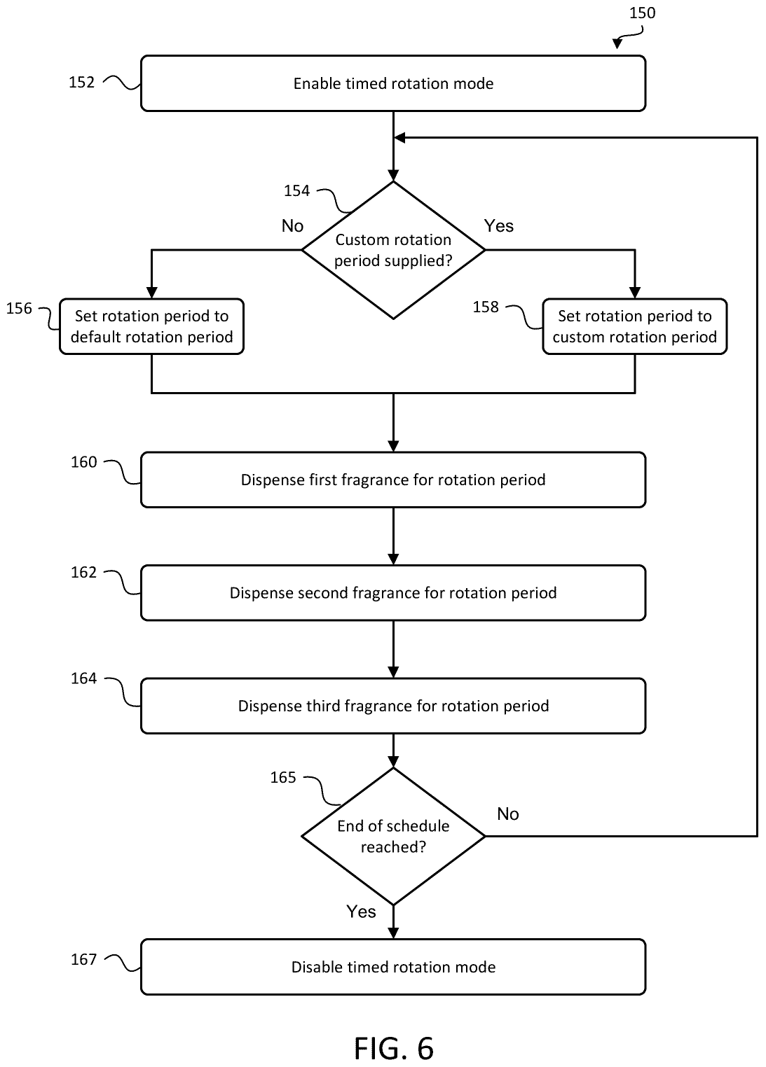

In the timed rotation mode, fragrances from multiple fragrance containers are individually and consecutively dispensed in a predictable order each according to a corresponding rotation period. The rotation period includes a set time for dispensing each one of the fragrances. More specifically, in the timed rotation mode, a first fragrance from a first one of multiple fragrance containers is dispensed for a first rotation period (see, e.g., block 160 of method 150 of FIG. 6) and at a desired intensity. At the end of the first rotation period, dispensing of the first fragrance from the first one of the multiple fragrance containers stops and a second fragrance from a second one of the multiple fragrance containers is dispensed for a second rotation period (equal to the first rotation period) (see, e.g., block 162 of the method 150 of FIG. 6) and at the desired intensity (which can be different or the same as the desired intensity of the first fragrance). At the end of the second rotation period, dispensing of the second fragrance from the second one of the multiple fragrance containers stops. If the fragrance diffuser 100 includes additional fragrance assemblies, a third fragrance from a third one of the multiple fragrance containers is dispensed for a third rotation period (equal to the first and second rotation periods) (see, e.g., block 164 of the method 150 of FIG. 6), at the desired intensity (which can be the same as the previous desired intensities or different) and so on until fragrances from all the fragrance containers are consecutively dispensed for the set time associated with the rotation periods. The order of the fragrances can be selected by a user and received by the timed rotation module 140 via the user rotation input 142. Moreover, although the rotation periods can be the same as presented above, in other examples, the rotation periods can be different, such as when a user desires a longer rotation period for a preferred fragrance over another.

As shown in FIG. 6, after the fragrances from all the fragrance containers are consecutively dispensed, the method 150 includes determining whether the end of a user-set schedule has been reached at block 165. If the end of the schedule has been reached, then the timed rotation mode is disabled at block 167 of the method 150. However, if the end of the schedule has not yet been reached, then the timed rotation mode remains enabled and the method 150 returns to start consecutively dispensing fragrances according to the rotation period. It is recognized that, in some examples, the step executed at block 165 can be continuously executed while the timed rotation mode is enabled so that as soon as the end of the schedule is reached the timed rotation mode is disabled regardless of completion of any one of the blocks 160-162.

In some examples, the timed rotation module 140 includes a default rotation period, which can be associated with olfactory fatigue for an average user and which may be adjustable in some examples. For example, the rotation period can be between, and inclusive of, 30 minutes and 3 hours (e.g., between, and inclusive of, 30 minutes and 1 hour). In certain examples, the user can select a custom rotation period that is different than the default rotation period. The custom rotation period is received by the timed rotation module 140 via the user rotation input 142. Accordingly, after the timed rotation mode is enabled at block 152 of the method 150, the method 150 determines whether a custom rotation period has been supplied by the user at block 154. If a custom rotation period is not supplied by the user, then the rotation period is set to the default rotation period at block 156. However, if a custom rotation period was supplied by the user, then the rotation period is set to the custom rotation period at block 158.

Because the fragrances of the fragrance diffuser 100 are consecutively rotated through each for a limited period of time (e.g., less than a period of time associated with olfactory fatigue), the user's nasal receptors do not become fatigued with any one fragrance. Accordingly, all fragrances diffused by the fragrance diffuser 100, when in the timed rotation mode, remain noticeable by the user, which increases the effectiveness of the fragrance diffuser 100 over conventional diffusers.

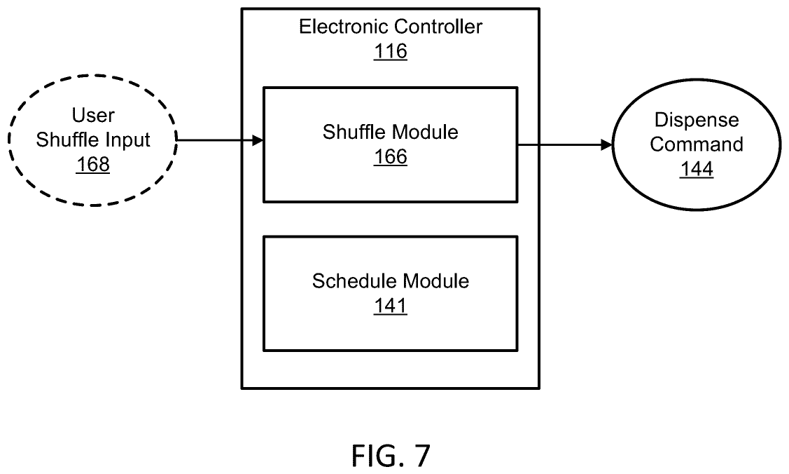

Referring to FIG. 7, according to some examples, the electronic controller 116 includes a shuffle module 166. The shuffle module 166 is configured to enable a shuffle mode of the fragrance diffuser 100 by generating the dispense command 144 pursuant to operations within the shuffle mode. As described above, in response to the dispense command 144, an intended dispenser dispenses the fragrance corresponding with the dispenser. The dispense command 144 is generated in response to user shuffle input 168 from a user. The user shuffle input 168 identifies a user's desire to enable (e.g., start) the shuffle mode of the fragrance diffuser 100. The user shuffle input 168 is received from the user via the user device 132 or the user interface 109. In some examples, the user shuffle input 168 also includes a desired schedule for operating the fragrance diffuser 100 in the shuffle mode. In response to receipt of a desired schedule, from the user shuffle input 168, the schedule module 141 is operable to run the fragrance diffuser 100 in the shuffle mode according to the desired schedule. For example, the desired schedule may include a start time and an end time and the schedule module 141 is configured to control the shuffle module 166 to start and stop generating the dispense command 144 according to the desired schedule. Additionally, in certain examples, the user shuffle input 168 also includes an intensity value, which corresponds with a desired intensity of the fragrances diffused in the shuffle mode. In some examples, the desired intensities for each one of the fragrances diffused in the shuffle mode can be different.

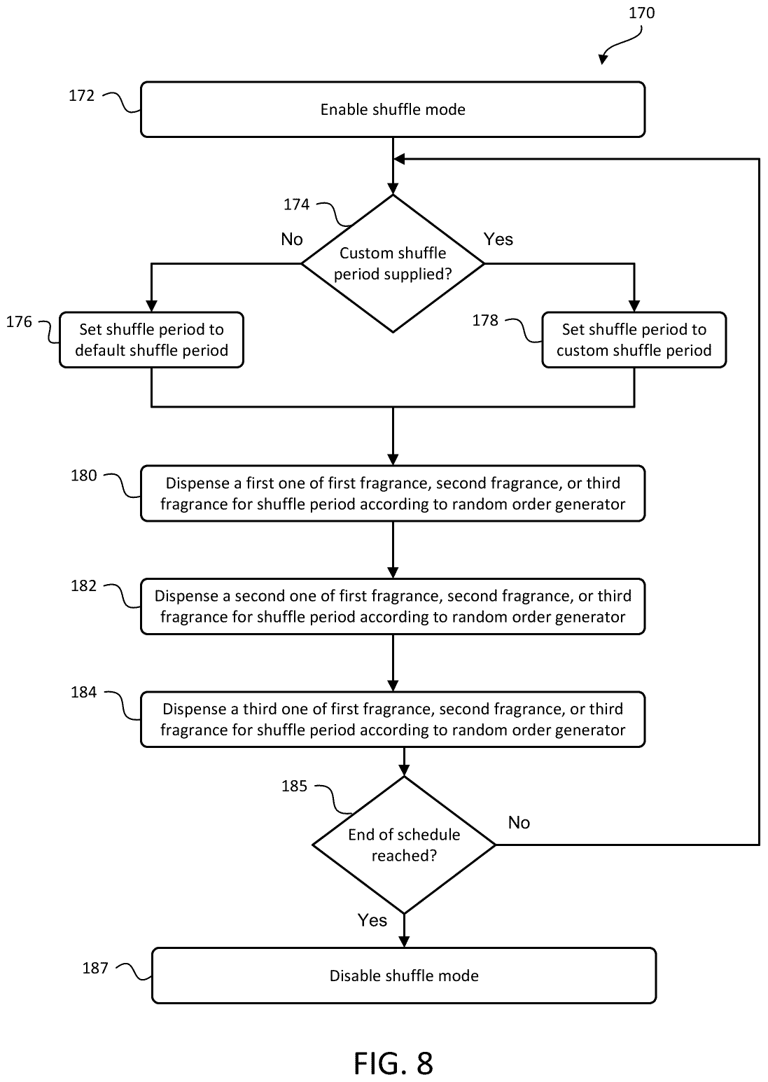

In the shuffle mode, fragrances from multiple fragrance containers are individually and consecutively dispensed in a random order each according to a corresponding shuffle period. The shuffle period includes a set time for dispensing each one of the fragrances in the random order. More specifically, in the shuffle mode, a first one of a first fragrance, a second fragrance, or a third fragrance, from a corresponding first one of a first fragrance container, a second fragrance container, or a third fragrance container, is dispensed for a first shuffle period (see, e.g., block 180 of method 170 of FIG. 8). The first one of the first fragrance, the second fragrance, or the third fragrance is randomly selected by a random order generator incorporated into the shuffle module 166. At the end of the first shuffle period, dispensing of the first one of the first fragrance, the second fragrance, or the third fragrance stops and a second one of the first fragrance, the second fragrance, or the third fragrance is dispensed for a second shuffle period (equal to the first shuffle period) (see, e.g., block 182 of the method 170 of FIG. 8). The second one of the first fragrance, the second fragrance, or the third fragrance is randomly selected by the random order generator of the shuffle module 166. At the end of the second shuffle period, dispensing of the second one of the first fragrance, the second fragrance, or the third fragrance stops. If the fragrance diffuser 100 includes additional fragrance assemblies, a third one of the first fragrance, the second fragrance, or the third fragrance is dispensed for a third shuffle period (equal to the first and second shuffle periods) (see, e.g., block 184 of the method 170 of FIG. 8), and so on until fragrances from all the fragrance containers are dispensed, in a random order, for the set time associated with the shuffle periods.

As shown in FIG. 8, after the fragrances from all the fragrance containers are randomly dispensed, the method 170 includes determining whether the end of a user-set schedule has been reached at block 185. If the end of the schedule has been reached, then the shuffle mode is disabled at block 187 of the method 170. However, if the end of the schedule has not yet been reached, then the shuffle mode remains enabled and the method 170 returns to start randomly dispensing fragrances according to the shuffle period. Accordingly, in some examples, as long as the schedule has not been reached and the shuffle mode has not been disabled or stopped, the fragrance diffuser 100 will shuffle through the fragrances for the shuffle periods in multiple cycles where each cycle has a different order than at least the previous cycle. For example, in a fragrance diffuser having three fragrances A, B, and C, in the shuffle mode, the fragrance diffuser can shuffle through cycles of the fragrances in a first random order B-A-C, then a second random order C-B-A, then a third random order A-C-B, etc. It is recognized that, in some examples, the step executed at block 185 can be continuously executed while the shuffle mode is enabled so that as soon as the end of the schedule is reached the shuffle mode is disabled regardless of completion of any one of the blocks 180-184.

In some examples, the shuffle module 166 includes a default shuffle period, which can be associated with olfactory fatigue for an average user, such as described above in association with the default rotation period. In other words, the default shuffle period can be the same as the default rotation period. In certain examples, the user can select a custom shuffle period that is different than the default shuffle period. The custom shuffle period is received by the shuffle module 166 via the user shuffle input 168. Accordingly, after the shuffle mode is enabled at block 172 of the method 170, the method 170 determines whether a custom shuffle period has been supplied by the user at block 174. If a custom shuffle period is not supplied by the user, then the shuffle period is set to the default shuffle period at block 176. However, if a custom shuffle period was supplied by the user, then the shuffle period is set to the custom shuffle period at block 178.

Similar to the timed rotation mode, in the shuffle mode, because the fragrances of the fragrance diffuser 100 are dispensed in a random order each for a limited period of time (e.g., less than a period of time associated with olfactory fatigue), the user's nasal receptors do not become fatigued with any one fragrance. Accordingly, all fragrances diffused by the fragrance diffuser 100, when in the shuffle mode, remain noticeable by the user.

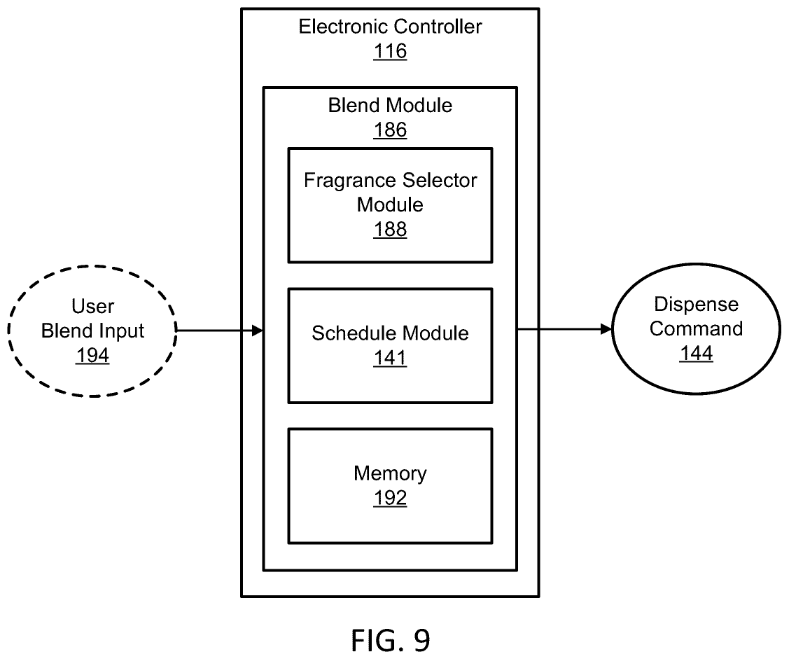

Referring to FIG. 9, according to some examples, the electronic controller 116 includes a blend module 186. The blend module 186 is configured to enable a blend mode of the fragrance diffuser 100 by generating the dispense command 144 pursuant to operations within the blend mode. As described above, when operating in the blend mode, in response to the dispense command 144 at least two intended dispensers concurrently dispense, and thus blend, fragrances, at desired intensities and for a desired blend period, corresponding with the intended dispensers. The dispense command 144 is generated in response to user blend input 194 from a user. The user blend input 194 identifies a user's desire to enable (e.g., start) the blend mode of the fragrance diffuser 100. Additionally, the user blend input 194 includes a desired blend of fragrances, which can be quantified as associated percentages of the fragrances to be blended and a total intensity of the blend of fragrances. Each fragrance of the blend of fragrances makes up a customizable percentage of the blend. For example, the user blend input 194 can include a first percentage of a first fragrance and a second percentage of a second fragrance, where the first and second percentages total 100% of the blend of fragrances. In another example, the user blend input 194 can include a first percentage of a first fragrance, a second percentage of a second fragrance, and a third percentage of a third fragrance, where the first, second, and third percentages total 100% of the blend of fragrances. Of course, in examples where the fragrance diffuser 100 contains more than three fragrances, the user blend input 194 can include percentages of more than three fragrances when quantifying the blend.

In some examples, the user blend input 194 also includes a blend intensity value, which corresponds with a desired overall intensity of the blend of fragrances. Based on the desired overall intensity of the blend of fragrances, the intensity of each one of the individual fragrances of the blend can be correspondingly (e.g., proportionally) adjusted to meet both the overall intensity of the blend and the desired percentages of the fragrances of the blend.

In response to the desired blend of fragrances and intensity of the blend, a fragrance selector module 188 of the blend module 186 generates a dispense command 144 for each one of the dispensers of the fragrances forming the blend of fragrances commensurate with the corresponding percentage of the fragrances and intensity of the blend. The percentage of a fragrance is determined by the rate of fragrance dispensed. Moreover, the rate that a fragrance is dispensed directly affects the intensity of the fragrance. Accordingly, dispensing a fragrance of a blend at a lower rate (or intensity), relative to the dispensing rates (or intensities) of other fragrances of the blend, results in the fragrance making up a lower percentage of the blend than the other fragrances. Moreover, a lower intensity of any one of the fragrances of a blend results in a lower overall intensity of the blend. In certain examples, the dispense command 144 controls the dispensing rate of a fragrance by varying the intensity of the electrical power signal. The dispensing rate of a fragrance can be proportional to the intensity of the electrical power signal associated with the dispense command 144. Accordingly, for a blend of fragrances having different percentages of each fragrance of the blend, the fragrance selector module 188 generates dispense commands 144 associated with electrical power signals having different intensities.

A user of the fragrance diffuser 100 can use the user device 132 or the user interface 109 to select any of various blends of fragrances contained within the fragrance diffuser 100. In some examples, the user inputs, via the user device 132 or the user interface 109, the particular fragrance in each of the fragrance containers of the fragrance diffuser 100 and a memory of the blend module 186 stores the particular fragrances. When the user selects a blend of fragrances, the blend can be saved in the memory 192 of the electronic controller 116 or a memory of the user device 132 (e.g., a cloud storage associated with the user device 132). The saved blend can then be re-selected at a later time for diffusion by the fragrance diffuser 100. Additionally, the saved blend can be shared with other users across a network of users (e.g., any of various social networks). In this manner, each individual user of a network can create and share new blends of fragrances with others, who in turn can replicate the blends on their own fragrance diffusers. In certain examples, each use of a custom blend of fragrances can be monitored within the network, and ratings/rankings can be applied to the blend of fragrances in comparison to other custom blends of fragrances.

In some examples, the user blend input 194 also includes a desired schedule for operating the fragrance diffuser 100 in the blend mode. In response to receipt of a desired schedule, from the user blend input 194, the schedule module 141 is operable to run the fragrance diffuser 100 in the blend mode according to the desired schedule. For example, the desired schedule may include a start time and an end time and the schedule module 141 is configured to control the blend module 186 to start and stop generating the dispense command 144 according to the desired schedule.

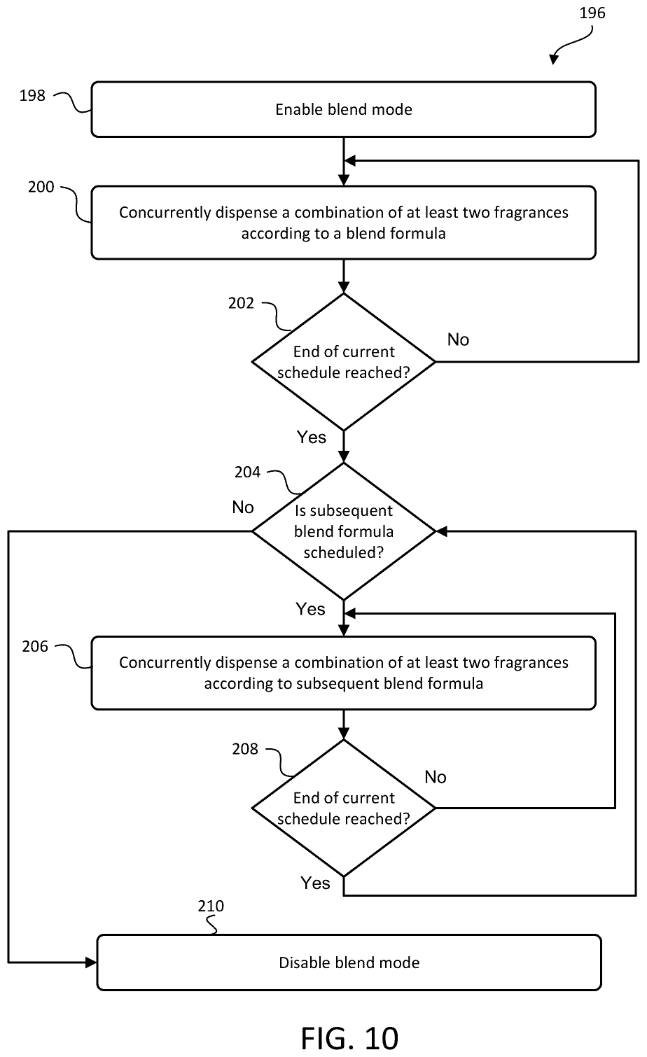

Referring to FIG. 10, when the blend mode is enabled at block 198 of method 196, fragrances from multiple fragrance containers are combined and concurrently dispensed according to a blend formula selected by a user, as indicated in block 200 of the method 196. The blend is dispensed indefinitely or until a current schedule is reached, as determined at block 202. After a current schedule is reached, the method 196 includes determining at block 204 whether a subsequent blend formula is scheduled to be dispensed at 204. If a subsequent blend formula is not scheduled, the method 196 proceeds to disable the blend mode at 210. However, if a subsequent blend formula is scheduled, the method 196 concurrently dispenses a combination of fragrances according to a subsequent blend formula at block 206 (and at an overall intensity that is the same as or different than the previous blend formula) indefinitely or until a current schedule is reached, as determined at block 208. Additional blends of fragrances are dispensed thereafter if scheduled. Based on the foregoing, in the blend mode, blends of fragrances, whether customized or predefined, can be dispensed consecutively according to a schedule or queue. In some examples, each schedule is set to a period of time less than the period of time associated with olfactory fatigue, so all the blends of fragrances diffused by the fragrance diffuser 100, when in the blend mode, remain noticeable by the user.

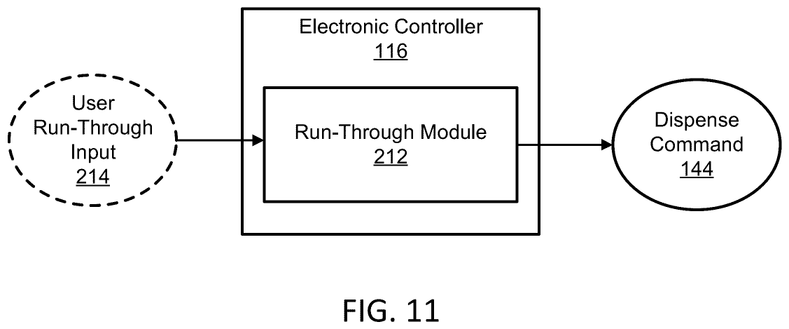

Referring to FIG. 11, according to some examples, the electronic controller 116 includes a run-through module 212. The run-through module 212 is configured to enable a run-through mode of the fragrance diffuser 100 by generating the dispense command 144 pursuant to operations within the run-through mode. As described above, in response to the dispense command 144 an intended dispenser dispenses the fragrance corresponding with the dispenser. The dispense command 144 is generated in response to user run-through input 214 from a user. The user run-through input 214 identifies a user's desire to enable (e.g., start) the run-through mode of the fragrance diffuser 100. The user run-through input 214 is received from the user via the user device 132 or the user interface 109. Additionally, in certain examples, the user run-through input 214 also includes an intensity value, which corresponds with a desired intensity of the fragrances diffused in the run-through mode. In some examples, the desired intensity of each one of the fragrances diffused in the run-through mode can be different.

In the run-through mode, fragrances from multiple fragrance containers are individually and consecutively fully dispensed in an order, one at a time, until all the fragrances are dispensed. In other words, the fragrance from a first one of multiple fragrance containers is fully dispensed, until the first one of the multiple fragrance containers is empty, before a subsequent one of the multiple fragrance containers is fully dispensed. The run-through mode continues until all the fragrances from all the fragrance containers are fully dispensed, or when the operational mode of the fragrance diffuser 100 is changed. In some examples, when the fragrance diffuser 100 is turned off while in the run-through mode, the fragrance diffuser 100 continues to operate in the run-through mode after the fragrance diffuser 100 is turned back on. Moreover, in certain examples, an intensity of the diffused fragrances can be adjusted during the run-through mode without disabling the run-through mode.

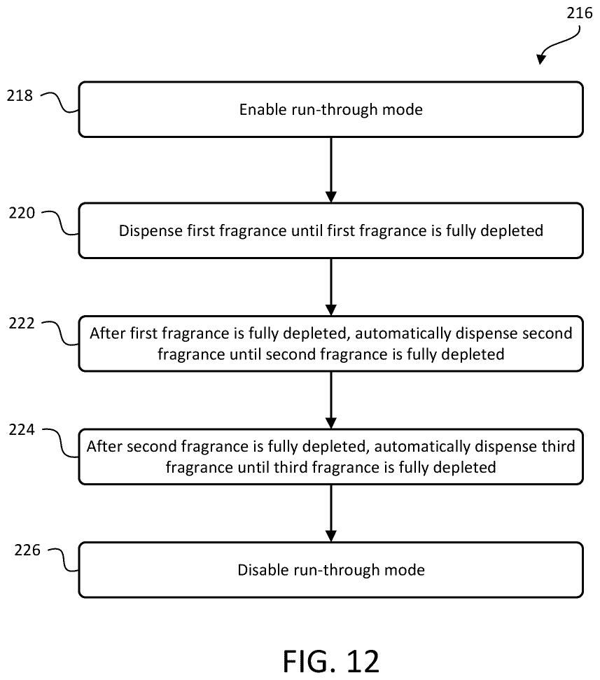

Referring to FIG. 12, for a fragrance diffuser 100 that includes at least three fragrance containers, a method 216 includes enabling the run-through mode at block 218 and dispensing a first fragrance from a first fragrance container until the first fragrance is fully depleted from the first fragrance container at block 220. After the first fragrance is fully depleted at block 220, the method 216 includes dispensing a second fragrance from a second fragrance container until the second fragrance is fully depleted from the second fragrance container at block 222. After the second fragrance is fully depleted at block 222, the method 216 includes dispensing a third fragrance from a third fragrance container until the third fragrance is fully depleted from the third fragrance container at block 224. These steps continue until the fragrance from all the fragrance containers of the fragrance diffuser 100 are depleted, at which point the method 216 disables the run-through mode at block 226. In some examples, the fragrance diffuser 100 determines the depletion of fragrance from a container via any of various methods, such as a physical sensor or a virtual sensor (e.g., an approximation via an algorithm).

Although the fragrance diffuser 100 in the illustrated examples includes three fragrance containers and three fragrances, and modes of operation of the fragrance diffuser 100 are described in association with three fragrances, it is recognized that, in some other examples, the fragrance diffuser 100 includes only two fragrance containers and two fragrances, or includes four or more fragrance containers and four or more fragrances, and the modes of operation can be practiced with two fragrance containers and two fragrances, or with four or more fragrance containers and fragrances. Additionally, as described above, the fragrance diffuser 100 can be configured to operate in one, two, three, or all four of the modes of operation disclosed herein. Therefore, in certain examples, although the electronic controller 116 is shown in FIGS. 5, 7, 9, and 11 as having a corresponding one of the timed rotation module 140, the shuffle module 166, the blend module 186, or the run-through module 212, in some examples, the electronic controller 116 of the fragrance diffuser 100 can include one, two, three, or all four of the timed rotation module 140, the shuffle module 166, the blend module 186, and the run-through module 212.

In preferred examples, when operating in the various modes described herein, the dispensing of a second or subsequent fragrance or fragrance blend occurs immediately following the stopping of the dispensing of the first or previous fragrance or fragrance blend, so that fragrances are substantially continuously diffused when operating in the various modes.

In the above description, certain terms may be used such as “up,” “down,” “upper,” “lower,” “horizontal,” “vertical,” “left,” “right,” “over,” “under” and the like. These terms are used, where applicable, to provide some clarity of description when dealing with relative relationships. But, these terms are not intended to imply absolute relationships, positions, and/or orientations. For example, with respect to an object, an “upper” surface can become a “lower” surface simply by turning the object over. Nevertheless, it is still the same object. Further, the terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise. Further, the term “plurality” can be defined as “at least two.” Moreover, unless otherwise noted, as defined herein a plurality of particular features does not necessarily mean every particular feature of an entire set or class of the particular features.

The term “about” or “substantially” in some embodiments, is defined to mean within +/−5% of a given value, however in additional embodiments any disclosure of “about” may be further narrowed and claimed to mean within +/−4% of a given value, within +/−3% of a given value, within +/−2% of a given value, within +/−1% of a given value, or the exact given value. Further, when at least two values of a variable are disclosed, such disclosure is specifically intended to include the range between the two values regardless of whether they are disclosed with respect to separate embodiments or examples, and specifically intended to include the range of at least the smaller of the two values and/or no more than the larger of the two values. Additionally, when at least three values of a variable are disclosed, such disclosure is specifically intended to include the range between any two of the values regardless of whether they are disclosed with respect to separate embodiments or examples, and specifically intended to include the range of at least the A value and/or no more than the B value, where A may be any of the disclosed values other than the largest disclosed value, and B may be any of the disclosed values other than the smallest disclosed value.

Additionally, instances in this specification where one element is “coupled” to another element can include direct and indirect coupling. Direct coupling can be defined as one element coupled to and in some contact with another element. Indirect coupling can be defined as coupling between two elements not in direct contact with each other, but having one or more additional elements between the coupled elements. Further, as used herein, securing one element to another element can include direct securing and indirect securing. Additionally, as used herein, “adjacent” does not necessarily denote contact. For example, one element can be adjacent to another element without being in contact with that element.

As used herein, the phrase “at least one of”, when used with a list of items, means different combinations of one or more of the listed items may be used and only one of the items in the list may be needed. The item may be a particular object, thing, or category. In other words, “at least one of” means any combination of items or number of items may be used from the list, but not all of the items in the list may be required. For example, “at least one of item A, item B, and item C” may mean item A; item A and item B; item B; item A, item B, and item C; or item B and item C. In some cases, “at least one of item A, item B, and item C” may mean, for example, without limitation, two of item A, one of item B, and ten of item C; four of item B and seven of item C; or some other suitable combination.

Unless otherwise indicated, the terms “first,” “second,” etc. are used herein merely as labels, and are not intended to impose ordinal, positional, or hierarchical requirements on the items to which these terms refer. Moreover, reference to, e.g., a “second” item does not require or preclude the existence of, e.g., a “first” or lower-numbered item, and/or, e.g., a “third” or higher-numbered item.

As used herein, a system, apparatus, structure, article, element, component, or hardware “configured to” perform a specified function is indeed capable of performing the specified function without any alteration, rather than merely having potential to perform the specified function after further modification. In other words, the system, apparatus, structure, article, element, component, or hardware “configured to” perform a specified function is specifically selected, created, implemented, utilized, programmed, and/or designed for the purpose of performing the specified function. As used herein, “configured to” denotes existing characteristics of a system, apparatus, structure, article, element, component, or hardware which enable the system, apparatus, structure, article, element, component, or hardware to perform the specified function without further modification. For purposes of this disclosure, a system, apparatus, structure, article, element, component, or hardware described as being “configured to” perform a particular function may additionally or alternatively be described as being “adapted to” and/or as being “operative to” perform that function.

The schematic flow chart diagrams included herein are generally set forth as logical flow chart diagrams. As such, the depicted order and labeled steps are indicative of one example of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

The electronic controller and any associated modules described in this specification may be implemented as a hardware circuit comprising custom VLSI circuits or gate arrays, off-the-shelf semiconductors such as logic chips, transistors, or other discrete components. The electronic controller may also be implemented in programmable hardware devices such as field programmable gate arrays, programmable array logic, programmable logic devices or the like.

The electronic controller may also be implemented in code and/or software for execution by various types of processors. An identified module of code may, for instance, comprise one or more physical or logical blocks of executable code which may, for instance, be organized as an object, procedure, or function. Nevertheless, the executables of the electronic controller need not be physically located together, but may comprise disparate instructions stored in different locations which, when joined logically together, comprise the electronic controller and achieve the stated purpose for the electronic controller.

Indeed, code of the electronic controller may be a single instruction, or many instructions, and may even be distributed over several different code segments, among different programs, and across several memory devices. Similarly, operational data may be identified and illustrated herein within the electronic controller, and may be embodied in any suitable form and organized within any suitable type of data structure. The operational data may be collected as a single data set, or may be distributed over different locations including over different computer readable storage devices. Where the electronic controller or portions of the electronic controller are implemented in software, the software portions are stored on one or more computer readable storage devices.

Any combination of one or more computer readable medium may be utilized. The computer readable medium may be a computer readable storage medium. The computer readable storage medium may be a storage device storing the code. The storage device may be, for example, but not limited to, an electronic, magnetic, optical, electromagnetic, infrared, holographic, micromechanical, or semiconductor system, apparatus, or device, or any suitable combination of the foregoing.

More specific examples (a non-exhaustive list) of the storage device would include the following: an electrical connection having one or more wires, a portable computer diskette, a hard disk, a random-access memory (RAM), a read-only memory (ROM), an erasable programmable read-only memory (EPROM or Flash memory), a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

Code for carrying out operations for embodiments may be written in any combination of one or more programming languages including an object-oriented programming language such as Python, Ruby, Java, Smalltalk, C++, or the like, and conventional procedural programming languages, such as the “C” programming language, or the like, and/or machine languages such as assembly languages. The code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

In the above description, numerous specific details are provided, which can include examples of programming, software modules, user selections, network transactions, database queries, database structures, hardware modules, hardware circuits, hardware chips, etc., to provide a thorough understanding of embodiments. One skilled in the relevant art will recognize, however, that embodiments may be practiced without one or more of the specific details, or with other methods, components, materials, and so forth. In other instances, well-known structures, materials, or operations are not shown or described in detail to avoid obscuring aspects of an embodiment.

Aspects of some examples are described above with reference to schematic flowchart diagrams and/or schematic block diagrams of methods, apparatuses, systems, and program products according to examples. It will be understood that each block of the schematic flowchart diagrams and/or schematic block diagrams, and combinations of blocks in the schematic flowchart diagrams and/or schematic block diagrams, can be implemented by code. This code may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the schematic flowchart diagrams and/or schematic block diagrams block or blocks.

The code may also be stored in a storage device that can direct a computer, other programmable data processing apparatus, or other devices to function in a particular manner, such that the instructions stored in the storage device produce an article of manufacture including instructions which implement the function/act specified in the schematic flowchart diagrams and/or schematic block diagrams block or blocks.

The code may also be loaded onto a computer, other programmable data processing apparatus, or other devices to cause a series of operational steps to be performed on the computer, other programmable apparatus or other devices to produce a computer implemented process such that the code which execute on the computer or other programmable apparatus provide processes for implementing the functions/acts specified in the flowchart and/or block diagram block or blocks.

The schematic flowchart diagrams and/or schematic block diagrams in the Figures illustrate the architecture, functionality, and operation of possible implementations of apparatuses, systems, methods and program products according to various embodiments. In this regard, each block in the schematic flowchart diagrams and/or schematic block diagrams may represent a module, segment, or portion of code, which comprises one or more executable instructions of the code for implementing the specified logical function(s).

It should also be noted that, in some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. Other steps and methods may be conceived that are equivalent in function, logic, or effect to one or more blocks, or portions thereof, of the illustrated Figures.

The present subject matter may be embodied in other specific forms without departing from its spirit or essential characteristics. The described examples are to be considered in all respects only as illustrative and not restrictive. Examples of the scope of the present subject matter can be found in the following numbered paragraphs.

1. A fragrance diffuser, comprising:
a housing configured to house at least two fragrance containers each containing a corresponding one of at least two fragrances;
at least two dispensers each configured to dispense a corresponding one of the at least two fragrances from a corresponding one of the at least two fragrance containers when the at least two fragrance containers are housed by the housing; and
an electronic controller configured to operate the fragrance diffuser in at least one of a timed rotation mode, a shuffle mode, a blend mode, or a run-through mode;
wherein, when the at least two fragrance containers are housed by the housing:
   in the timed rotation mode, the electronic controller controls the at least two dispensers so that (i) a first one of the at least two dispensers dispenses a first one of the at least two fragrances for a first rotation period; and (ii) after the first rotation period, a second one of the at least two dispensers dispenses a second one of the at least two fragrances for a second rotation period;
   in the shuffle mode, the electronic controller controls the at least two dispensers so that (i) the first one of the at least two dispensers dispenses the first one of the at least two fragrances for a first shuffle period, wherein the first one of the at least two dispensers is randomly selected from the at least two dispensers via a random order generator of the electronic controller; and (ii) after the first shuffle period, the second one of the at least two dispensers dispenses the second one of the at least two fragrances for a second shuffle period equal to the first shuffle period;
   in the blend mode, the electronic controller controls the at least two dispensers so that (i) concurrently, the first one of the at least two dispensers dispenses the first one of the at least two fragrances and the second one of the at least two dispensers dispenses the second one of the at least two fragrances; and (ii) the first one of the at least two fragrances, after being dispensed, blends with the second one of the at least two fragrances, after being dispensed, to form a blend of fragrances; and
   in the run-through mode, the electronic controller controls the at least two dispensers so that (i) the first one of the at least two dispensers dispenses the first one of the at least two fragrances until the first one of the at least two fragrances is depleted from the first one of the at least two dispensers; and (ii) after the first one of the at least two fragrances is depleted from the first one of the at least two dispensers, the second one of the at least two dispensers automatically dispenses the second one of the at least two fragrances until the second one of the at least two fragrances is depleted from the second one of the at least two dispensers.

2. The fragrance diffuser according to claim 1, wherein the electronic controller is configured to operate the fragrance diffuser in at least two of the timed rotation mode, the shuffle mode, the blend mode, or the run-through mode.

3. The fragrance diffuser according to claim 2, wherein the electronic controller is configured to operate the fragrance diffuser in at least three of the timed rotation mode, the shuffle mode, the blend mode, or the run-through mode.

4. The fragrance diffuser according to claim 3, wherein the electronic controller is configured to operate the fragrance diffuser in all four of the timed rotation mode, the shuffle mode, the blend mode, and the run-through mode.

5. The fragrance diffuser according to claim 1, wherein the electronic controller is configured to operate the fragrance diffuser in at least the timed rotation mode.

6. The fragrance diffuser according to claim 5, wherein each one of the first rotation period and the second rotation period is equal to or less than a time period associated with olfactory fatigue.

7. The fragrance diffuser according to claim 5, wherein the first rotation period and the second rotation period is a user-selected one of a plurality of rotation periods.

8. The fragrance diffuser according to claim 5, wherein the first one of the at least two dispensers is a user-selected one of the at least two dispensers.

9. The fragrance diffuser according to claim 5, wherein, in the time rotation mode, the electronic controller is configured to continuously switch between dispensing the at least two fragrances for a scheduled period of time.

10. The fragrance diffuser according to claim 5, wherein the second rotation period is equal to the first rotation period.

11. The fragrance diffuser according to claim 5, wherein the second rotation period is different than the first rotation period.

12. The fragrance diffuser according to claim 1, wherein the electronic controller is configured to operate the fragrance diffuser in at least the shuffle mode.

13. The fragrance diffuser according to claim 12, wherein each one of the first shuffle period and the second shuffle period is equal to or less than a time period associated with olfactory fatigue.

14. The fragrance diffuser according to claim 12, wherein the first shuffle period and the second shuffle period is a user-selected one of a plurality of shuffle periods.

15. The fragrance diffuser according to claim 12, wherein, in the shuffle mode, the electronic controller is configured to continuously switch between dispensing the at least two fragrances in a random order for a scheduled period of time.

16. The fragrance diffuser according to claim 15, wherein, in the shuffle mode, the fragrance diffuser is configured to dispense the at least two fragrances in multiple cycles, and wherein an order of the at least two fragrances in a first one of the multiple cycles is different than the order of the at least two fragrances in a second one of the multiple cycles.

17. The fragrance diffuser according to claim 1, wherein the electronic controller is configured to operate the fragrance diffuser in at least the blend mode.

18. The fragrance diffuser according to claim 17, wherein:
the first one of the at least two fragrances makes up a first percentage of the blend of fragrances;
the second one of the at least two fragrances makes up a second percentage of the blend of fragrances; and
each one of the first percentage and the second percentage is a user-selected one of a plurality of percentages.

19. The fragrance diffuser according to claim 18, wherein, when operating in the blend mode:
the electronic controller is configured to diffuse the first one of the at least two fragrances at a first intensity and to diffuse the second one of the at least two fragrances at a second intensity; and
the first intensity of the first one of the at least two fragrances and the second intensity of the second one of the at least two fragrances are combined to equal a user-selected one of a plurality of intensities.

20. The fragrance diffuser according to claim 18, wherein:
the electronic controller further comprises a memory; and
the blend of fragrances, including the first percentage of the first one of the at least two fragrances and the second percentage of the second one of the at least two fragrances, is stored in the memory after the first percentage and the second percentage are selected by a user.

21. The fragrance diffuser according to claim 20, wherein the blend of fragrances stored in the memory is re-selectable by a user.

22. The fragrance diffuser according to claim 20, wherein the fragrance diffuser is configured to share with others, across a computer network, the blend of fragrances, including the first percentage of the first one of the at least two fragrances and the second percentage of the second one of the at least two fragrances, selected by the user.

23. The fragrance diffuser according to claim 17, wherein, in the blend mode:
the electronic controller is further configured to control the at least two dispensers so that concurrently, the first one of the at least two dispensers dispenses the first one of the at least two fragrances and the second one of the at least two dispensers dispenses the second one of the at least two fragrances form a second blend of fragrances different than the blend of fragrances; and
the electronic controller is configured to continuously switch between dispensing the blend of fragrances, for a blend period, and the second blend of fragrances, for the blend period, for a scheduled period of time.

24. The fragrance diffuser according to claim 23, wherein the blend period is equal to or less than a time period associated with olfactory fatigue.

25. The fragrance diffuser according to claim 1, wherein the electronic controller is configured to operate the fragrance diffuser in at least the run-through mode.

26. The fragrance diffuser according to claim 1, wherein:
the housing is configured to house at least three fragrance containers each containing a corresponding one of at least three fragrances;
the fragrance diffuser comprises at least three dispensers each configured to dispense a corresponding one of the at least three fragrances from a corresponding one of the at least three fragrance containers when the at least three fragrance containers are housed by the housing;
when the at least three fragrance containers are housed by the housing:
   in the timed rotation mode, the electronic controller controls the at least three dispensers so that (i) the first one of the at least three dispensers dispenses the first one of the at least three fragrances for the first rotation period; (ii) after the first rotation period, the second one of the at least three dispensers dispenses the second one of the at least three fragrances for the second rotation period; and (iii) after the second rotation period, a third one of the at least three dispensers dispenses a third one of the at least three fragrances for a third rotation period equal to the second rotation period;
   in the shuffle mode, the electronic controller controls the at least three dispensers so that (i) the first one of the at least three dispensers dispenses the first one of the at least three fragrances for the first shuffle period, wherein the first one of the at least three dispensers is randomly selected from the at least three dispensers via the random order generator of the electronic controller; (ii) after the first shuffle period, the second one of the at least three dispensers dispenses the second one of the at least three fragrances for the second shuffle period; and (iii) after the second shuffle period, a third one of the at least three dispensers dispenses a second one of the at least three fragrances for a third shuffle period equal to the second shuffle period;
   in the blend mode, the electronic controller controls the at least three dispensers so that (i) concurrently, the first one of the at least three dispensers dispenses the first one of the at least three fragrances, the second one of the at least three dispensers dispenses the second one of the at least three fragrances, and a third one of the at least three dispensers dispenses a third one of the at least three fragrances; and (ii) the first one of the at least three fragrances, after being dispensed, blends with the second one of the at least two fragrances, after being dispensed, and blends with the third one of the at least three fragrances, to form the blend of fragrances; and
   in the run-through mode, the electronic controller controls the at least three dispensers so that (i) the first one of the at least three dispensers dispenses the first one of the at least three fragrances until the first one of the at least three fragrances is depleted from the first one of the at least three dispensers; (ii) after the first one of the at least three fragrances is depleted from the first one of the at least three dispensers, the second one of the at least three dispensers automatically dispenses the second one of the at least three fragrances until the second one of the at least three fragrances is depleted from the second one of the at least three dispensers; and (iii) after the second one of the at least three fragrances is depleted from the second one of the at least three dispensers, a third one of the at least three dispensers automatically dispenses a third one of the at least three fragrances until the third one of the at least three fragrances is depleted from the third one of the at least three dispensers.

27. The fragrance diffuser according to claim 1, wherein the electronic controller comprises a schedule module configured to operate the fragrance diffuser in each one of the at least one of the timed rotation mode, the shuffle mode, the blend mode, or the run-through mode according to a user-selected one of a plurality of schedules.

28. The fragrance diffuser according to claim 1, wherein the electronic controller is configured to diffuse the first one of the at least two fragrances and the second one of the at least two fragrances according to a user-selected one of a plurality of intensities when the fragrance diffuser is operating in the at least one of the timed rotation mode, the shuffle mode, the blend mode, or the run-through mode.

29. A method of diffusing fragrances, comprising at least one of:
a timed rotation step comprising electronically controlling at least two dispensers so that (i) a first one of the at least two dispensers dispenses a first one of at least two fragrances for a first rotation period; and (ii) after the first rotation period, a second one of the at least two dispensers dispenses a second one of the at least two fragrances for a second rotation period;
a shuffle step comprising electronically controlling the at least two dispensers so that (i) the first one of the at least two dispensers dispenses the first one of the at least two fragrances for a first shuffle period, wherein the first one of the at least two dispensers is randomly selected from the at least two dispensers via a random order generator of the electronic controller; and (ii) after the first shuffle period, the second one of the at least two dispensers dispenses the second one of the at least two fragrances for a second shuffle period equal to the first shuffle period;
a blend step comprising electronically controlling the at least two dispensers so that (i) concurrently, the first one of the at least two dispensers dispenses the first one of the at least two fragrances and the second one of the at least two dispensers dispenses the second one of the at least two fragrances; and (ii) the first one of the at least two fragrances, after being dispensed, blends with the second one of the at least two fragrances, after being dispensed, to form a blend of fragrances; or
a run-through step comprising electronically controlling the at least two dispensers so that (i) the first one of the at least two dispensers dispenses the first one of the at least two fragrances until the first one of the at least two fragrances is depleted from the first one of the at least two dispensers; and (ii) after the first one of the at least two fragrances is depleted from the first one of the at least two dispensers, the second one of the at least two dispensers automatically dispenses the second one of the at least two fragrances until the second one of the at least two fragrances is depleted from the second one of the at least two dispensers.

30. The method according to claim 29, wherein the method comprises at least two of the timed rotation step, the shuffle step, the blend step, or the run-through step.

31. The method according to claim 29, wherein the method comprises at three of the timed rotation step, the shuffle step, the blend step, or the run-through step.

32. The method according to claim 29, wherein the method comprises all four of the timed rotation step, the shuffle step, the blend step, and the run-through step.

33. The method according to claim 29, wherein:
the method includes the timed rotation step;
each one of the first rotation period and the second rotation period is equal to or less than a time period associated with olfactory fatigue; and
the time rotation step further comprises selecting the first rotation period and the second rotation period from a plurality of rotation periods.

34. The method according to claim 29, wherein:
the method includes the shuffle step;
each one of the first shuffle period and the second shuffle period is equal to or less than a time period associated with olfactory fatigue;
the shuffle step further comprises selecting the first shuffle period and the second shuffle period from a plurality of shuffle periods; and
the shuffle step further comprises, for a scheduled period of time, switching between at least a first cycle, where the at least two fragrances are dispensed in a first order, and at least a second cycle, where the at least two fragrances are dispensed in a second order different than the first order.

35. The method according to claim 29, wherein:
the method includes the blend step;
the blend step further comprises selecting a first percentage and a second percentage from a plurality of percentages;
the first one of the at least two fragrances makes up the first percentage of the blend of fragrances; and
the second one of the at least two fragrances makes up the second percentage of the blend of fragrances.

36 The method according to claim 35, further comprising electronically storing the blend of fragrances to create a stored blend of fragrances, wherein the blend step further comprises:
selecting the stored blend of fragrances; and
electronically controlling the at least two dispensers so that (i) concurrently, the first one of the at least two dispensers dispenses the first one of the at least two fragrances and the second one of the at least two dispensers dispenses the second one of the at least two fragrances; and (ii) the first one of the at least two fragrances, after being dispensed, blends with the second one of the at least two fragrances, after being dispensed, to form the stored blend of fragrances.

37. The method according to claim 36, further comprising sharing the stored blend of fragrances with others across a computer network.

38. The method according to claim 35, wherein:
the blend step further comprises continuously switching between dispensing the blend of fragrances, for a blend period, and a second blend of fragrances, for the blend period, for a scheduled period of time;
the second blend of fragrances comprises a third percentage of the first one of the at least two fragrances and a fourth percentage of the second one of the at least two fragrances; and at least one of (i) the third percentage is different than the first percentage, or (ii) the fourth percentage is different than the second percentage.

39. The method according to claim 29, wherein the method includes the run-through step.

40. The method according to claim 29, further comprising selecting an intensity from a plurality of intensities, wherein each one of the timed rotation step, the shuffle step, the blend step, and the run-through step comprises dispensing the first one of the at least two fragrances and dispensing the second one of the at least two fragrances according to the selected intensity.

1. A fragrance diffuser, comprising:
a housing configured to house at least two fragrance containers each containing a corresponding one of at least two fragrances;
at least two dispensers each configured to dispense a corresponding one of the at least two fragrances from a corresponding one of the at least two fragrance containers when the at least two fragrance containers are housed by the housing; and
an electronic controller configured to operate the fragrance diffuser in at least one of a timed rotation mode, a shuffle mode, a blend mode, or a run-through mode;
wherein, when the at least two fragrance containers are housed by the housing:
in the timed rotation mode, the electronic controller controls the at least two dispensers so that (i) a first one of the at least two dispensers dispenses a first one of the at least two fragrances for a first rotation period; and (ii) after the first rotation period, a second one of the at least two dispensers dispenses a second one of the at least two fragrances for a second rotation period;
in the shuffle mode, the electronic controller controls the at least two dispensers so that (i) the first one of the at least two dispensers dispenses the first one of the at least two fragrances for a first shuffle period, wherein the first one of the at least two dispensers is randomly selected from the at least two dispensers via a random order generator of the electronic controller; and (ii) after the first shuffle period, the second one of the at least two dispensers dispenses the second one of the at least two fragrances for a second shuffle period equal to the first shuffle period;
in the blend mode, the electronic controller controls the at least two dispensers so that (i) concurrently, the first one of the at least two dispensers dispenses the first one of the at least two fragrances and the second one of the at least two dispensers dispenses the second one of the at least two fragrances; and (ii) the first one of the at least two fragrances, after being dispensed, blends with the second one of the at least two fragrances, after being dispensed, to form a blend of fragrances; and
in the run-through mode, the electronic controller controls the at least two dispensers so that (i) the first one of the at least two dispensers dispenses the first one of the at least two fragrances until the first one of the at least two fragrances is depleted from the first one of the at least two dispensers; and (ii) after the first one of the at least two fragrances is depleted from the first one of the at least two dispensers, the second one of the at least two dispensers automatically dispenses the second one of the at least two fragrances until the second one of the at least two fragrances is depleted from the second one of the at least two dispensers.

2. The fragrance diffuser according to claim 1, wherein the electronic controller is configured to operate the fragrance diffuser in at least two of the timed rotation mode, the shuffle mode, the blend mode, or the run-through mode.

3. The fragrance diffuser according to claim 2, wherein the electronic controller is configured to operate the fragrance diffuser in at least three of the timed rotation mode, the shuffle mode, the blend mode, or the run-through mode.

4. The fragrance diffuser according to claim 3, wherein the electronic controller is configured to operate the fragrance diffuser in all four of the timed rotation mode, the shuffle mode, the blend mode, and the run-through mode.

5. The fragrance diffuser according to claim 1, wherein the electronic controller is configured to operate the fragrance diffuser in at least the timed rotation mode;
each one of the first rotation period and the second rotation period is equal to or less than a time period associated with olfactory fatigue; and
the first rotation period and the second rotation period is a user-selected one of a plurality of rotation periods.

6.-8. (canceled)

9. The fragrance diffuser according to claim 5, wherein, in the time rotation mode, the electronic controller is configured to continuously switch between dispensing the at least two fragrances for a scheduled period of time.

10.-11. (canceled)

12. The fragrance diffuser according to claim 1, wherein the electronic controller is configured to operate the fragrance diffuser in at least the shuffle mode.

13. (canceled)

14. (canceled)

15. The fragrance diffuser according to claim 12, wherein, in the shuffle mode, the electronic controller is configured to continuously switch between dispensing the at least two fragrances in a random order for a scheduled period of time, and wherein each one of the first shuffle period and the second shuffle period is equal to or less than a time period associated with olfactory fatigue.

16. The fragrance diffuser according to claim 15, wherein, in the shuffle mode, the fragrance diffuser is configured to dispense the at least two fragrances in multiple cycles, and wherein an order of the at least two fragrances in a first one of the multiple cycles is different than the order of the at least two fragrances in a second one of the multiple cycles.

17. The fragrance diffuser according to claim 1, wherein the electronic controller is configured to operate the fragrance diffuser in at least the blend mode;
the first one of the at least two fragrances makes up a first percentage of the blend of fragrances;
the second one of the at least two fragrances makes up a second percentage of the blend of fragrances;
each one of the first percentage and the second percentage is a user-selected one of a plurality of percentages;
the electronic controller further comprises a memory; and
the blend of fragrances, including the first percentage of the first one of the at least two fragrances and the second percentage of the second one of the at least two fragrances, is stored in the memory after the first percentage and the second percentage are selected by a user.

18.-21. (canceled)

22. The fragrance diffuser according to claim 17, wherein the fragrance diffuser is configured to share with others, across a computer network, the blend of fragrances, including the first percentage of the first one of the at least two fragrances and the second percentage of the second one of the at least two fragrances, selected by the user.

23.-24. (canceled)

25. The fragrance diffuser according to claim 1, wherein the electronic controller is configured to operate the fragrance diffuser in at least the run-through mode.

26. (canceled)

27. The fragrance diffuser according to claim 1, wherein the electronic controller comprises a schedule module configured to operate the fragrance diffuser in each one of the at least one of the timed rotation mode, the shuffle mode, the blend mode, or the run-through mode according to a user-selected one of a plurality of schedules.

28. The fragrance diffuser according to claim 1, wherein the electronic controller is configured to diffuse the first one of the at least two fragrances and the second one of the at least two fragrances according to a user-selected one of a plurality of intensities when the fragrance diffuser is operating in the at least one of the timed rotation mode, the shuffle mode, the blend mode, or the run-through mode.

29. A method of diffusing fragrances, comprising at least one of:
a timed rotation step comprising electronically controlling at least two dispensers so that (i) a first one of the at least two dispensers dispenses a first one of at least two fragrances for a first rotation period; and (ii) after the first rotation period, a second one of the at least two dispensers dispenses a second one of the at least two fragrances for a second rotation period;
a shuffle step comprising electronically controlling the at least two dispensers so that (i) the first one of the at least two dispensers dispenses the first one of the at least two fragrances for a first shuffle period, wherein the first one of the at least two dispensers is randomly selected from the at least two dispensers via a random order generator of the electronic controller; and (ii) after the first shuffle period, the second one of the at least two dispensers dispenses the second one of the at least two fragrances for a second shuffle period equal to the first shuffle period;
a blend step comprising electronically controlling the at least two dispensers so that (i) concurrently, the first one of the at least two dispensers dispenses the first one of the at least two fragrances and the second one of the at least two dispensers dispenses the second one of the at least two fragrances; and (ii) the first one of the at least two fragrances, after being dispensed, blends with the second one of the at least two fragrances, after being dispensed, to form a blend of fragrances; or
a run-through step comprising electronically controlling the at least two dispensers so that (i) the first one of the at least two dispensers dispenses the first one of the at least two fragrances until the first one of the at least two fragrances is depleted from the first one of the at least two dispensers; and (ii) after the first one of the at least two fragrances is depleted from the first one of the at least two dispensers, the second one of the at least two dispensers automatically dispenses the second one of the at least two fragrances until the second one of the at least two fragrances is depleted from the second one of the at least two dispensers.

30.-32. (canceled)

33. The method according to claim 29, wherein:
the method includes the timed rotation step;
each one of the first rotation period and the second rotation period is equal to or less than a time period associated with olfactory fatigue; and
the time rotation step further comprises selecting the first rotation period and the second rotation period from a plurality of rotation periods.

34. The method according to claim 29, wherein:
the method includes the shuffle step;
each one of the first shuffle period and the second shuffle period is equal to or less than a time period associated with olfactory fatigue;
the shuffle step further comprises selecting the first shuffle period and the second shuffle period from a plurality of shuffle periods; and
the shuffle step further comprises, for a scheduled period of time, switching between at least a first cycle, where the at least two fragrances are dispensed in a first order, and at least a second cycle, where the at least two fragrances are dispensed in a second order different than the first order.

35. The method according to claim 29, wherein:
the method includes the blend step;
the blend step further comprises selecting a first percentage and a second percentage from a plurality of percentages;
the first one of the at least two fragrances makes up the first percentage of the blend of fragrances; and
the second one of the at least two fragrances makes up the second percentage of the blend of fragrances.

36.-37. (canceled)

38. The method according to claim 35, wherein:
the blend step further comprises continuously switching between dispensing the blend of fragrances, for a blend period, and a second blend of fragrances, for the blend period, for a scheduled period of time;
the second blend of fragrances comprises a third percentage of the first one of the at least two fragrances and a fourth percentage of the second one of the at least two fragrances; and
at least one of (i) the third percentage is different than the first percentage, or (ii) the fourth percentage is different than the second percentage.

39. The method according to claim 29, wherein the method includes the run-through step.

40. (canceled)
